(19) United States
(12) Patent Application Publication
(10) Pub. No.: US 2009/0082681 A1
(43) Pub. Date: Mar. 26, 2009

(54) BIOLOGICAL INFORMATION PROCESSING APPARATUS AND BIOLOGICAL INFORMATION PROCESSING METHOD

(75) Inventors: Sachie Yokoyama, Kanagawa (JP); Takuji Suzuki, Kanagawa (JP); Kazushige Ouchi, Saitama (JP)

Correspondence Address: OBLON, SPIVAK, MCCLELLAND MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 12/208,769

(22) Filed: Sep. 11, 2008

(30) Foreign Application Priority Data
Sep. 21, 2007 (JP) 2007-245222

(51) Int. Cl. A61B 5/0402; A61B 5/103

(52) U.S. Cl. 600/509; 600/595

(57) ABSTRACT

A biological information processing apparatus obtains a pulse wave signal indicating a pulse wave of a subject, and acceleration measured according to body motion of the subject and calculates an amount of body motion of the subject using the acceleration. By using at least one of the body motion amount and the acceleration, the apparatus approximates a heart rate of the subject and sets a parameter to be used for detection of a pulse interval using the heart rate. Then, the apparatus detects each pulse interval using a pulse waveform indicated by the pulse wave signal and the parameter.

### CROSS-REFERENCE TO RELATED APPLICATIONS

This application is based upon and claims the benefit of priority from the prior Japanese Patent Application No. 2007-245222, filed on Sep. 21, 2007; the entire contents of which are incorporated herein by reference.

### BACKGROUND OF THE INVENTION

1. Field of the Invention

The present invention relates to a biological information processing apparatus and a biological information processing method for measuring heartbeats based on a pulse wave or electrocardiogram to detect each heartbeat interval.

2. Description of the Related Art

A technique of detecting each interval of heartbeats as a pulse interval or a heartbeat interval based on a pulse waveform measured by a sphygmograph or a waveform of an electrocardiogram measured by an electrocardiograph is typically employed. The detected interval is subjected to frequency analysis, and resultant frequency components indicate activities of autonomic nerves such as sympathetic nerves and parasympathetic nerves. From the activities of the autonomic nerves, subsidiary information such as a stress level of a user, a quality of sleep including REM sleep and non-REM sleep, and an exercise load can be obtained. There are many types of sphygmographs and heart rate meters to be used to obtain the pulse interval and the heartbeat interval, respectively. For example, some heart rate meters are worn on a body trunk of a user, and some are worn on a wrist. Some sphygmographs are put on an ear of a user, and some sphygmographs utilize a photoplethysmographic sensor and are put on a wrist. Such sphygmographs are readily used, while motion of the user easily makes a pulse waveform erratic. Therefore, such sphygmographs are mostly used for measurement during rest. Recently, a technique of eliminating the influence of body motion from the pulse wave measured by such a sphygmograph is proposed (JP-A 2005-160640 (KOKAI)).

There is also a pulse-wave measuring apparatus that detects a pulse interval for measurement of an exercise load during an exercise. This type of pulse-wave measuring apparatus performs a process of recognizing a condition (exercise condition) of a user doing an exercise such as walking and jogging using an acceleration, and obtaining an average heart rate during the exercise, or the like. This type of pulse-wave measuring apparatus, however, cannot detect the pulse interval for each pulse so that it is unsuitable for applications of performing autonomic nerve analysis, such as calculating a stress level based on frequency analysis of fluctuation components of the pulse interval. In addition, the types of exercises done by the user whose condition can be recognized using the acceleration are limited to waling, jogging, and the like. Thus, in such a state that a user is doing an exercise other than waling and jogging in the daily life, the load of the exercise is difficult to measure.

The exercises to be performed in the daily life include for example going up and down of stairs and brisk walking. The pulse tends to be quickened immediately after such an exercise. When information of a pulse wave immediately after such an exercise can be obtained, this helps measurement of an exercise load in the daily life. In measuring the exercise load in the daily life, there is a risk of an erratic pulse waveform due to body motion, whereas it is useful to increase accuracy in detection of a pulse interval at rest during which no body motion occurs. However, during rest immediately after an exercise or between exercises, amplitude or baseline of the pulse wave greatly varies due to influences of the exercise performed immediately before. Thus, it is difficult to detect the pulse interval at high accuracy. Also a heartbeat interval obtained from an electrocardiogram measured by the electrocardiograph is difficult to detect at rest immediately after an exercise.

### SUMMARY OF THE INVENTION

According to one aspect of the present invention, a biological information processing apparatus includes an obtaining unit that obtains a pulse wave signal indicating a pulse wave of a subject and an acceleration measured according to body motion of the subject; a body-motion calculating unit that calculates an amount of body motion of the subject using the acceleration; an approximating unit that approximates a heart rate of the subject using at least one of the body motion amount and the acceleration; a setting unit that sets a parameter to be used for detection of a pulse interval, using the heart rate; and a detecting unit that detects each pulse interval using a pulse waveform indicated by the pulse wave signal and the parameter.

According to another aspect of the present invention, a biological information processing apparatus includes an obtaining unit that obtains an electrocardiograph signal indicating an electrocardiogram of a subject and an acceleration measured according to body motion of the subject; a body-motion calculating unit that calculates an amount of body motion of the subject using the acceleration; an approximating unit that approximates a heart rate of the subject using at least one of the body motion amount and the acceleration; a setting unit that sets a parameter to be used for detection of a heart rate interval, using the heart rate; and a detecting unit that detects each heart rate interval using an electrocardiogram waveform indicated by the electrocardiograph signal and the parameter.

According to still another aspect of the present invention, a biological-information processing method performed by a biological information processing apparatus including an obtaining unit, a body-motion calculating unit, an approximating unit, a setting unit, and a detecting unit, the method includes obtaining a pulse wave signal indicating a pulse wave of a subject, and an acceleration measured according to body motion of the subject, by the obtaining unit; calculating an amount of body motion of the subject using the acceleration, by the body-motion calculating unit; approximating a heart rate of the subject using at least one of the body motion amount and the acceleration, by the approximating unit; setting a parameter to be used for detection of a pulse interval using the heart rate, by the setting unit; and detecting each pulse interval using a pulse waveform indicated by the pulse wave signal and the parameter, by the detecting unit.

According to still another aspect of the present invention, a biological-information processing method performed by a biological information processing apparatus including an obtaining unit, a body-motion calculating unit, an approximating unit, a setting unit, and a detecting unit, the method includes obtaining an electrocardiograph signal indicating an electrocardiogram of a subject, and an acceleration measured according to body motion of the subject, by the obtaining unit; calculating an amount of body motion of the subject using the acceleration, by the body-motion calculating unit; approximating a heart rate of the subject using at least one of the body motion amount and the acceleration, by the approximating unit; setting a parameter to be used for detection of a heart rate interval using the heart rate, by the setting unit; and detecting each heart rate interval using an electrocardiogram waveform indicated by the electrocardiograph signal and the parameter, by the detecting unit.

### BRIEF DESCRIPTION OF THE DRAWINGS

FIG. 1 is a drawing illustrating a configuration of a biological information processing apparatus according to an embodiment of the present invention;

FIG. 2 is a drawing illustrating an example of an overview of the biological information processing apparatus and a state of placement thereof;

FIG. 3 is a drawing schematically illustrating a configuration of a pulse-wave measuring unit;

FIG. 4 is a drawing illustrating an example of the biological information processing apparatus having the pulse-wave measuring unit on a downside thereof;

FIG. 5 is a drawing illustrating an example of the biological information processing apparatus as shown in FIG. 4, being placed on a user's wrist like a wristwatch;

FIG. 6 is another example of the biological information processing apparatus having a form that can be placed on a user's ear;

FIG. 7 is a drawing illustrating an example of a data configuration of an exercise-intensity correspondence table;

FIG. 8 is a drawing illustrating an example of a data configuration of an individual information table;

FIG. 9 is a drawing illustrating an example of a data configuration of a factor table;

FIG. 10 is still another example of the biological information processing apparatus having a display unit on a front face thereof;

FIG. 11 is a flowchart of a pulse-interval detecting process procedure performed by the biological information processing apparatus;

FIG. 12 is a flowchart of a process procedure of approximating a heart rate;

FIG. 13 is a flowchart of a process procedure of calculating a rest start time, a rest end time, and an exercise end time;

FIG. 14 is a drawing illustrating an example of a relationship between an exercise end time and a great-change occurrence time;

FIG. 15 is a flowchart of a process procedure of detecting a pulse interval;

FIG. 16 is a drawing illustrating an example of a pulse wave from a most recent sampling time up to a setting time (during a time window);

FIG. 17 is a drawing illustrating an example of approximation of threshold value crossing;

FIG. 18 is a drawing illustrating an example of display of pulse interval data that is displayed on the display unit;

FIG. 19 is a drawing illustrating a state of a pulse wave when a user shifts from an exercise state to a rest state;

FIG. 20 is a drawing illustrating an example of a data configuration of an exercise-detail correspondence table;

FIG. 21 is a drawing illustrating an example of a data configuration of a second exercise-intensity correspondence table;

FIG. 22 is a flowchart of a process procedure of approximating a heart rate for explaining details of a process at one step according to a modification of the embodiment of the present invention;

FIG. 23 is another flowchart of a process procedure of approximating a heart rate for explaining details of a process at one step according to another modification of the embodiment;

FIG. 24 is a drawing illustrating an example of a data configuration of a normal range table according to still another modification of the embodiment;

FIG. 25 is a flowchart of a process procedure of determining whether a pulse interval according to the modification of the embodiment is erroneous;

FIG. 26 is a drawing illustrating an example of a configuration of a biological information processing apparatus according to still another modification of the embodiment;

FIG. 27 is a drawing illustrating an example of a configuration of a biological information processing apparatus according to still another modification of the embodiment, and a configuration of a biological-information measuring apparatus as an external device; and

FIG. 28 is a drawing illustrating an example of a configuration of a biological information processing apparatus according to still another modification of the embodiment, and a configuration of another biological-information measuring apparatus as an external device.

### DETAILED DESCRIPTION OF THE INVENTION

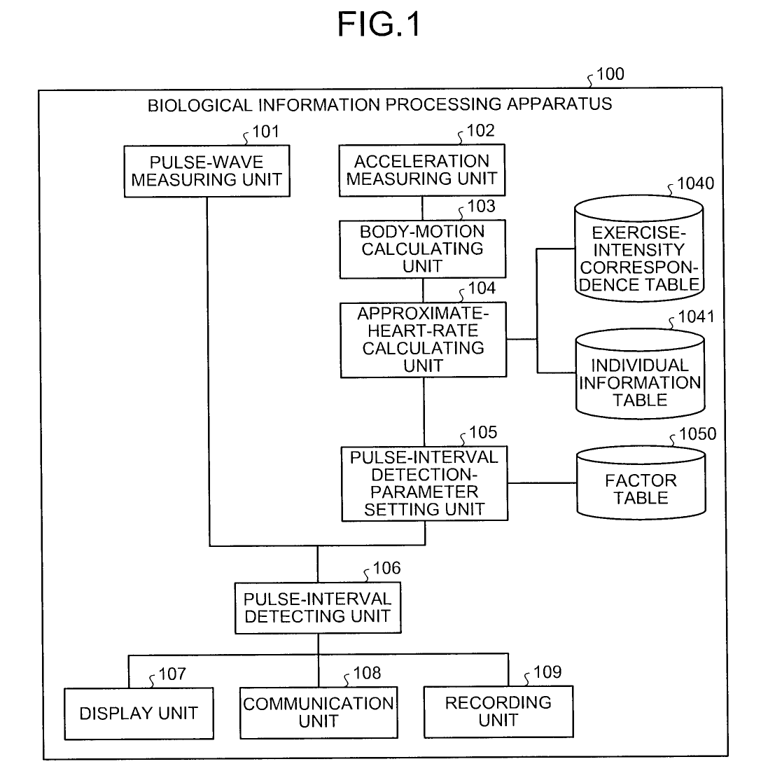

FIG. 1 is a drawing illustrating a configuration of a biological information processing apparatus 100 according to an embodiment of the present invention. As shown in FIG. 1, the biological information processing apparatus 100 includes a pulse-wave measuring unit 101, an acceleration measuring unit 102, a body-motion calculating unit 103 an approximate-heart-rate calculating unit 104, a pulse-interval detection-parameter setting unit 105, a pulse-interval detecting unit 106, a display unit 107, a communication unit 108, a recording unit 109, an exercise-intensity correspondence table 1040, an individual information table 1041, and a factor table 1050.

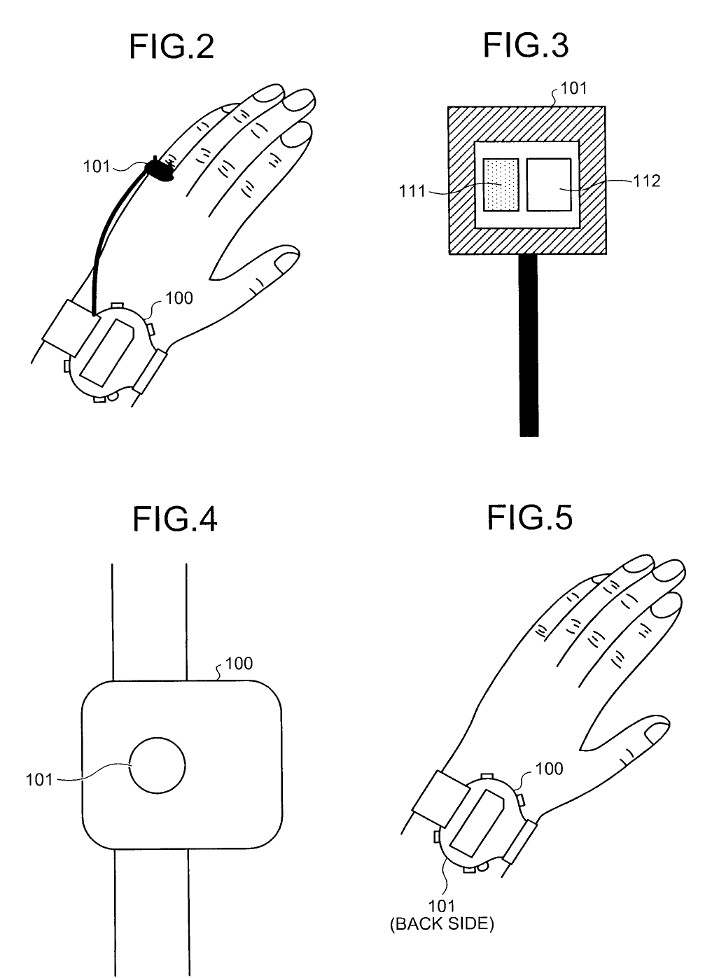

FIG. 2 is a drawing illustrating an example of an overview of the biological information processing apparatus 100 and a state of placement thereof. In this example, the biological information processing apparatus 100 is placed on a user's wrist like a wristwatch, and the pulse-wave measuring unit 101 is put on a finger. A pulse wave is measured on a palmar surface of the finger, and a pulse wave signal indicating the measured the pulse wave is outputted.

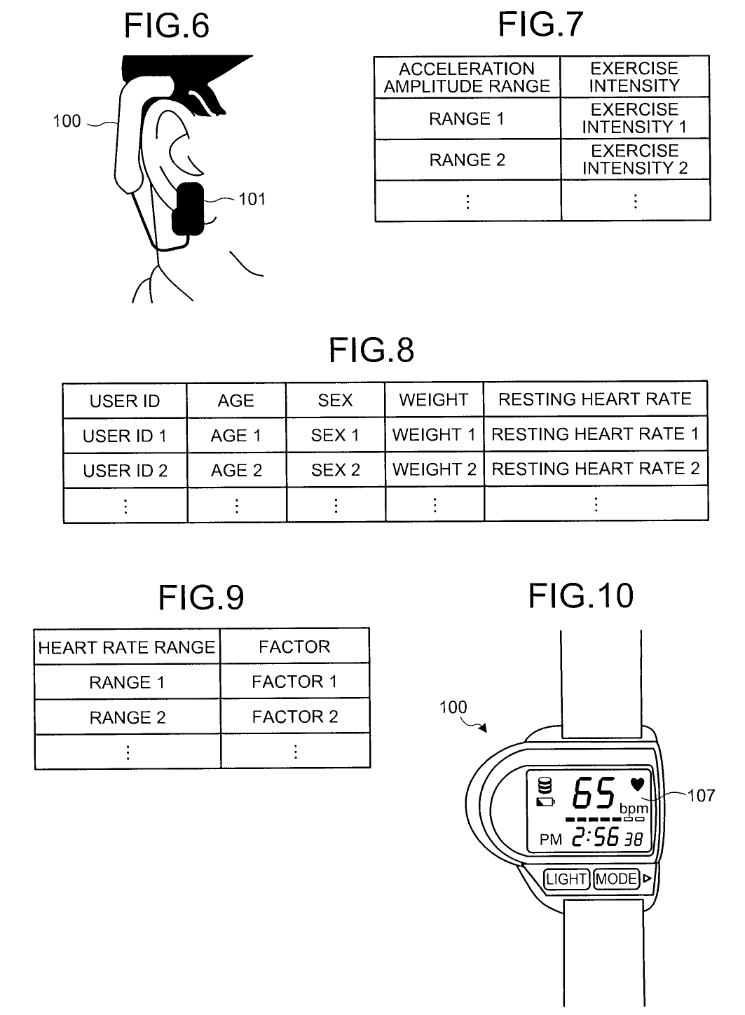

FIG. 3 is a drawing schematically illustrating a configuration of the pulse-wave measuring unit 101. A photoplethysmographic sensor including a combination of a light-emitting diode (LED) 111 and a photodiode 112 is mounted on the pulse-wave measuring unit 101. In the pulse-wave measuring unit 101, the LED 111 applies light to the user's skin, and the photodiode 112 detects changes in intensity of reflected light (which can be transmitted light) due to changes in blood flow, thereby obtaining a pulse wave. Thus, the pulse-wave measuring unit 101 measures the pulse wave and outputs a pulse wave signal indicating the measured the pulse wave. As the color of the LED 111, blue, green, red, or near infrared, which is well absorbed by blood hemoglobin, is employed. The photodiode 112 having characteristics corresponding to a waveband of the LED 111 that is used is preferably selected. FIG. 4 is a drawing illustrating an example of the biological information processing apparatus 100 having the pulse-wave measuring unit 101 on a side facing a wrist when the biological information processing apparatus 100 is placed on a user's wrist. FIG. 5 is a drawing illustrating an example of the biological information processing apparatus 100 as shown in FIG. 4, which is placed on a user's wrist like a wristwatch. In this example, a pulse wave is measured on the wrist. The pulse-wave measuring unit 101 in this example can include the photoplethysmographic sensor that is configured by the combination of the LED 111 and the photodiode 112 as shown in FIG. 3, or can include a pressure sensor that obtains changes in arterial pulse using pressure. FIG. 6 is another example of the biological information processing apparatus 100 having a form that can be placed on a user's ear. In this example, the pulse-wave measuring unit 101 is placed on an ear lobule for measurement of the pulse wave. The pulse-wave measuring unit 101 in this example preferably includes a photoplethysmographic sensor being configured by the combination of the LED 111 and the photodiode 112 as shown in FIG. 3.

Returning to FIG. 1, the acceleration measuring unit 102 includes an acceleration sensor that measures an acceleration. The acceleration sensor is placed on a predetermined site of the user, and the acceleration measuring unit 102 measures acceleration according to user's body motion and outputs the measured acceleration. The acceleration sensor can measure the acceleration in one axial direction, or can measure the accelerations for example in three directions of X, Y, and Z axes. While there are many types of acceleration sensors such as a piezoresistive type, a piezoelectric type, and a capacitance type, any type of acceleration sensor can be used to detect the acceleration.

The biological information processing apparatus 100 obtains the pulse wave signal outputted from the pulse-wave measuring unit 101 and the acceleration outputted from the acceleration measuring unit 102 via an input port (not shown) being obtaining means as hardware.

The body-motion calculating unit 103 calculates an amount of body motion using the acceleration outputted from the acceleration measuring unit 102. A method of calculating an amount of body motion using the acceleration is described for example in JP-A 2001-344352 (KOKAI).

FIG. 7 is a drawing illustrating an example of a data configuration of the exercise-intensity correspondence table 1040 (first correspondence information). The exercise-intensity correspondence table 1040 stores therein a correspondence relation previously set between exercise intensity and amplitude of the acceleration. FIG. 8 is a drawing illustrating an example of a data configuration of the individual information table 1041. The Individual information table 1041 previously stores therein individual information of users, being related to corresponding user's IDs. The individual information includes a user's ID, age, sex, weight, and a heart rate at rest (resting heart rate) of the user. The approximate-heart-rate calculating unit 104 calculates an exercise time period using the acceleration outputted from the acceleration measuring unit 102 and the body motion amount calculated by the body-motion calculating unit 103. The approximate-heart-rate calculating unit 104 then obtains exercise intensity by referring to the exercise-intensity correspondence table 1040 based on the acceleration measured and outputted during the exercise time period. The approximate-heart-rate calculating unit 104 then calculates an approximate heart rate using the obtained exercise intensity, a maximum heart rate calculated based on the individual information stored in the individual information table 1041, and the resting heart rate stored in the individual information table 1041, as approximation of the heart rate.

The correspondence relation between the exercise intensity and the amplitude range of the acceleration stored in the exercise-intensity correspondence table 1040 is for example described in the following reference literature 1.
(Reference Literature 1) An attempt to the volume of exercise measurement using a portable accelerometer, Tomohiro Tanikawa, Kawasaki medical welfare journal, Vol. 11, No. 2, 2001, pp. 313 to 318

The maximum heart rate can be calculated for example by a Karvonen method. This is for example described in the following reference literature 2. The maximum heart rate can be calculated upon each calculation of the approximate heart rate, or can be previously calculated based on the individual information as mentioned above and stored in the individual information table 1041.
(Reference Literature 2) Science of heart rate for exercise prescription, Keiji Yamaji, 1981, Taishukan

A method of obtaining an approximate heart rate using the exercise intensity, the maximum heart rate, and the resting heart rate is described for example in the following reference literature 3.
(Reference Literature 3) A comparative study for estimate of energy expenditure, Akira Takushima, Journal of health science, Vol. 9, pp. 137 to 145, 1987

The factor table 1050 (fourth correspondence information) stores therein a correspondence relation between factors to be used for calculation of a setting time (which is described later) that will be used in detecting a pulse interval, and ranges of heart rate. FIG. 9 is a drawing illustrating an example of a data configuration of the factor table 1050. In this example, the correspondence relation between the heart rate ranges and the factors is set so that a shorter setting time is calculated for a range of higher heart rates while a longer setting time is calculated for a range of lower heart rates. The pulse-interval detection-parameter setting unit 105 obtains a factor with reference to the factor table 1050 using the approximate heart rate calculated by the approximate-heart-rate calculating unit 104, and calculates the setting time using the obtained factor. That is, the pulse-interval detection-parameter setting unit 105 sets the setting time as a parameter to be used in detecting the pulse interval.

The pulse-interval detecting unit 106 includes a filter like a finite impulse response (FIR) filter, a low-pass filter (LPF), or a high-pass filter (HPF). The pulse-interval detecting unit 106 samples the pulse signal outputted from the pulse-wave measuring unit 101, eliminates noise components (including noises and fluctuations of a baseline) from the pulse signal other than the pulse wave, performs signal processing like steepening of the pulse waveform, and then detects a pulse interval. A method of detecting a pulse wave is described for example in JP-A 2001-344352 (KOKAI). More specifically, for example, the pulse-interval detecting unit 106 updates a maximum value and a minimum value of a pulse wave from a most recent sampling time up to the setting time (that is, during a time window), and sets a median of the maximum value and the minimum value as a pulse-interval detection threshold value. The pulse-interval detecting unit 106 determines whether the pulse wave crosses the pulse-interval detection threshold value, thereby detecting a candidate for the pulse interval. The pulse-interval detecting unit 106 determines whether the detected candidate for the pulse interval is within a predetermined pulse interval range, and detects the pulse interval based on a result of the determination.

In the present embodiment, the pulse-interval detecting unit 106 uses the setting time calculated by the pulse-interval detection-parameter setting unit 105. However, the setting time for a resting time is set at 1.5 seconds based on a standard pulse rate of 60 beats per minute (bpm). The pulse interval (second) is obtained by dividing the pulse rate (bpm) by 60 seconds.

The display unit 107 includes a display such as a liquid crystal display (LCD). The display unit 107 displays data such as data of the pulse interval detected by the pulse-interval detecting unit 106 (pulse interval data), the pulse signal outputted by the pulse-wave measuring unit 101, or the body motion amount calculated by the body-motion calculating unit 103. FIG. 10 is a drawing illustrating an example of the biological information processing apparatus 100 having the display unit 107 on its front face.

The recording unit 109 is a storage area that stores therein various measurement data measured by the biological information processing apparatus 100. The recording unit 109 includes for example a flash memory, or an electrically erasable programmable read-only memory (EEPROM). The measurement data include the pulse wave signal, the body motion amount, the pulse interval data, and the like.

The communication unit 108 transfers the measurement data to an external terminal with wireless (electromagnetic or optical) communication such as Bluetooth and infrared communication, or wired communication such as a universal serial bus (USB) and a Recommended Standard 232 version C (RS-232C). The communication unit 108 can transfer the measurement data upon each measurement of the data, or can transfer collection of the measurement data accumulated in the recording unit 109.

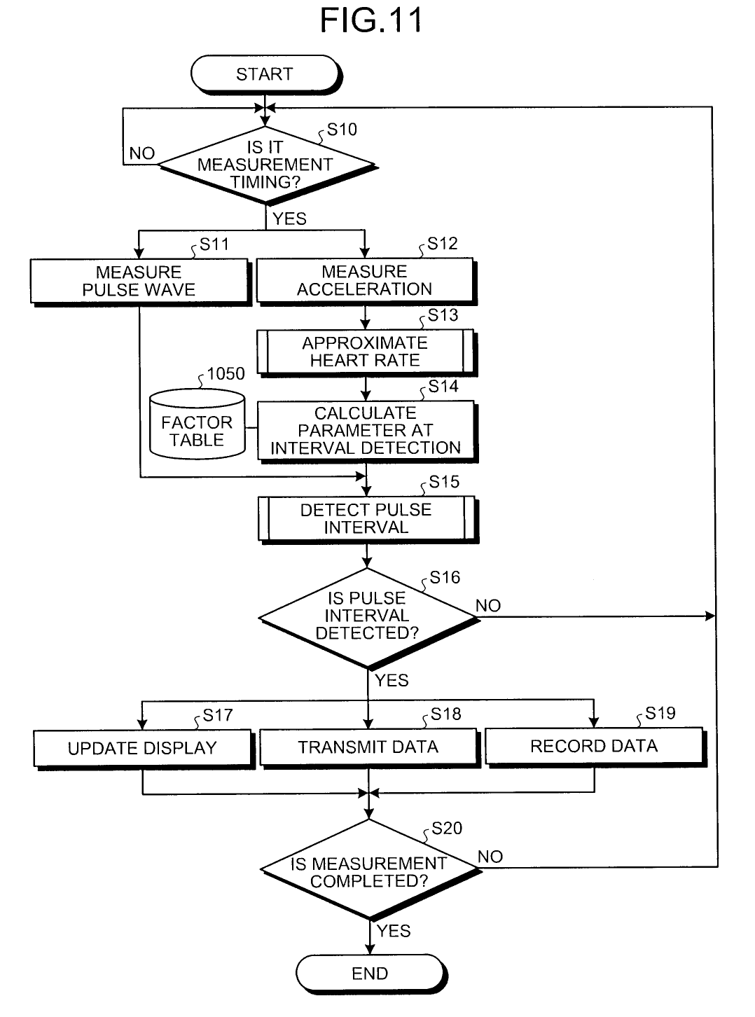

An operation of the biological information processing apparatus 100 according to the present embodiment is explained next. FIG. 11 is a flowchart of a pulse-interval detecting process procedure performed by the biological information processing apparatus 100. An example in which the biological information processing apparatus 100 is placed on a user's wrist as shown in FIG. 2 or 5 is explained. When a user operates a power switch or an operation button (neither shown) of the biological information processing apparatus 100 to instruct to start measuring a pulse wave, the pulse-wave measuring unit 101 measures a pulse wave in a predetermined sampling cycle, and outputs a pulse signal indicating the measured pulse wave. The sampling cycle is for example 50 milliseconds. When a sampling timing comes in this sampling cycle (YES at step S10), the biological information processing apparatus 100 outputs a pulse signal using the pulse-wave measuring unit 101 (step S11). The biological information processing apparatus 100 also outputs acceleration using the acceleration measuring unit 102 (step S12). The biological information processing apparatus 100 approximates a heart rate using the approximate-heart-rate calculating unit 104 (step S13).

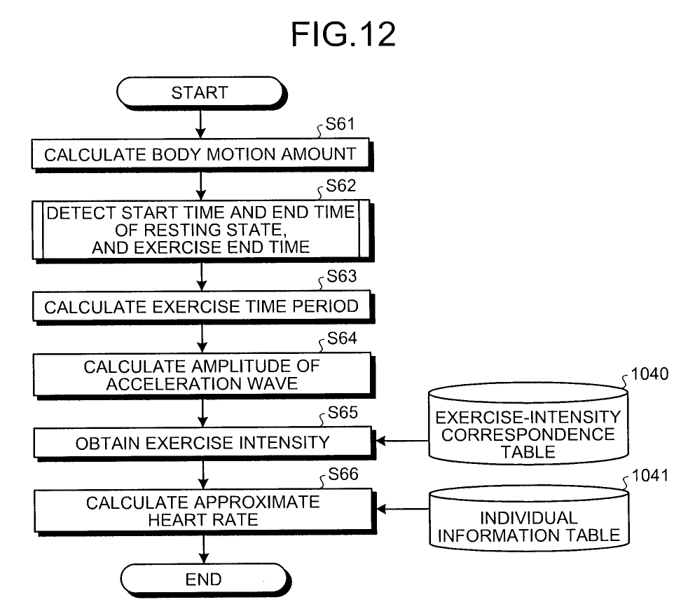

A detailed process procedure at step S13 is explained. FIG. 12 is a flowchart of a process procedure of approximating a heart rate. The body-motion calculating unit 103 calculates an amount of body motion using the acceleration outputted by the acceleration measuring unit 102 at step S12 in FIG. 11 (step S61). The approximate-heart-rate calculating unit 104 then determines whether the user is in a resting state or exercising state based on the calculated body motion amount, and calculates a start point of a resting state (rest start time), an end point of the resting state (rest end time), and an end point of an exercising state (exercise end time) (step S62). The approximate-heart-rate calculating unit 104 then calculates an exercise time period from a start point of an exercising state up to the end point of the exercising state, using the rest start time, the rest end time, and the exercise end time (step S63). Details of the process at step S62 are explained later.

The approximate-heart-rate calculating unit 104 then calculates amplitude of the acceleration wave using the acceleration measured and outputted by the acceleration measuring unit 102 during the exercise time calculated at step S63 (step S64). The approximate-heart-rate calculating unit 104 then obtains exercise intensity corresponding to the amplitude calculated at step S64, with reference to the exercise-intensity correspondence table 1040 (step S65). The approximate-heart-rate calculating unit 104 calculates the an approximate heart rate as approximation of the heart rate using the obtained exercise intensity, the resting heart rate stored in the individual information table 1041, and a maximum heart rate calculated based on the individual information stored in the individual information table 1041 (step S66). For example, assume that the amplitude of the acceleration wave is 4.5 G/s after the user walks continuously for one minute at 3 km/h, and that the exercise intensity (%VO2max) corresponding thereto is 30%. Assuming that the heart rate at rest is 60 bpm and the maximum heart rate is 190 bpm, an approximate heart rate obtained by the method as described in the reference literature 3 is 69 bpm.

If there is no time when the user is in an exercising state and thus no exercise time period is calculated at step S63, the approximate-heart-rate calculating unit 104 sets the approximate heart rate for example at 60 bpm, which is equal to the heart rate at rest.

To specify the individual information to be used at step S66, the user ID is employed. For example, the user can operate an operation button and input the user ID in instructing to start measuring a pulse wave, whereby the biological information processing apparatus 100 can obtain the user ID. Alternatively, the user can input the user ID via an operation button for example at initial setting, so that the user ID can be stored in a storage unit (not shown) in the biological information processing apparatus 100. The biological information processing apparatus 100 can obtain the user ID by reading the user ID from the storage unit when performing the process at step S66.

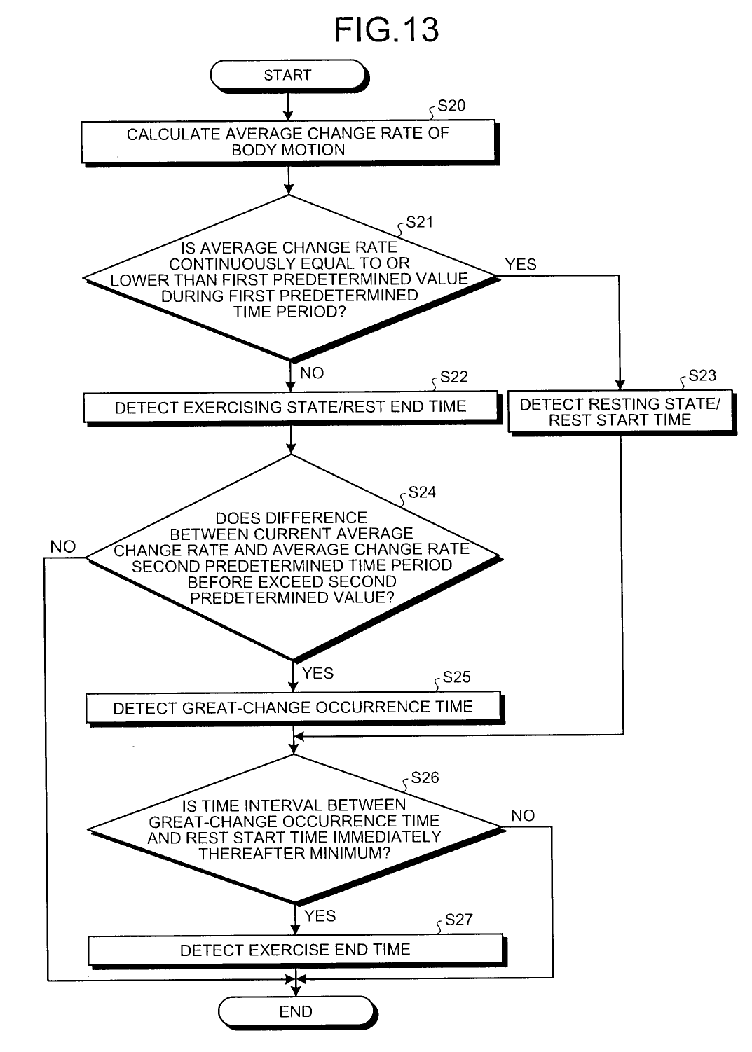

A detailed process procedure at step S62 is explained next. FIG. 13 is a flowchart of a process procedure of calculating the rest start time, the rest end time, and the exercise end time. The approximate-heart-rate calculating unit 104 calculates an average change rate of the body motion amount calculated at step S61 (step S20), and determines whether the average change rate is continuously equal to or lower than a first predetermined value during a first predetermined time period (for example, two seconds) (step S21). When a result of the determination at step S21 is YES, the approximate-heart-rate calculating unit 104 determines that the user is during a resting state, and detects this point in time as the rest start time (step S23). When a result of the determination at step S21 is No, the approximate-heart-rate calculating unit 104 determines that the user is during an exercising state, and detects this point in time as the rest end time (step S22). When determining that the user is during an exercising state, the approximate-heart-rate calculating unit 104 determines whether a difference between an average change rate calculated at step S20 at the current time and an average change rate calculated at step S20 a second predetermined time period (for example, three seconds) before exceeds a second predetermined value (for example, 0.2G) (step S24). When a result of the determination at step S24 is YES, the approximate-heart-rate calculating unit 104 detects a time at this point as a time when great change in the body motion amount occurs (great-change occurrence time) (step S25). A plurality of the great-change occurrence times can be detected during an exercising state. The approximate-heart-rate calculating unit 104 determines whether a time interval between one of the great-change occurrence times and the rest start time detected at step S23 is minimum (step S26). When a result of the determination at step S26 is YES, the approximate-heart-rate calculating unit 104 detects the determined great-change occurrence time as the exercise end time (step S27). That is, at step S27, the approximate-heart-rate calculating unit 104 detects a time when grate change occurs in the body motion amount most recently before start of the resting state, as the exercise end time.

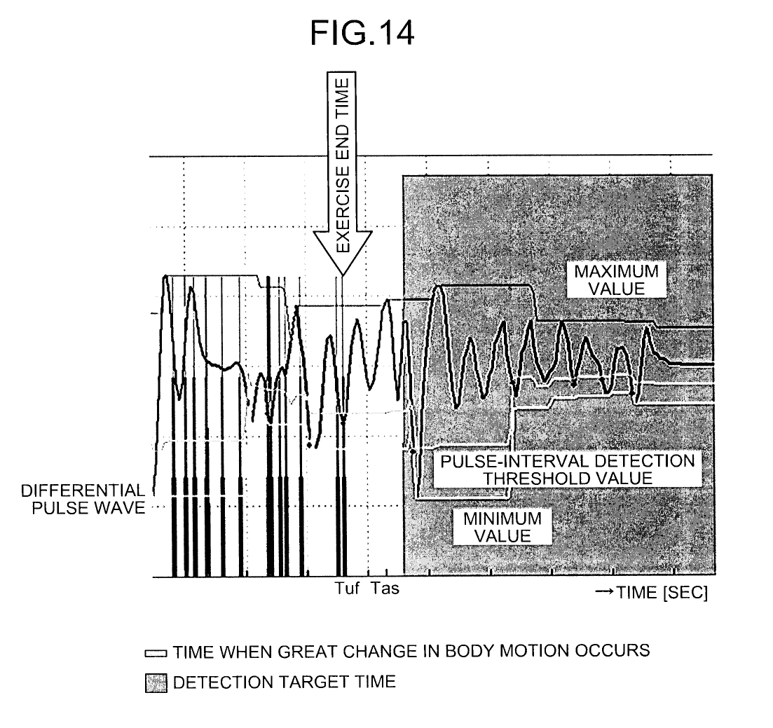

FIG. 14 is a drawing illustrating an example of a relation between the exercise end time and the great-change occurrence time. FIG. 14 indicates that plural great-change occurrence times are detected, and that one of the great-change occurrence times detected most recently before a rest start time Tas is detected as an exercise end time Tuf.

Return to the explanation of the pulse-interval detecting process with reference to FIG. 11. After step S13, the biological information processing apparatus 100 calculates a setting time to be used for detection of a pulse interval, using the pulse-interval detection-parameter setting unit 105 (step S14). The pulse-interval detection-parameter setting unit 105 obtains a factor corresponding to the approximate heart rate calculated by the approximate-heart-rate calculating unit 104 at step S13, with reference to the factor table 1050. The pulse-interval detection-parameter setting unit 105 then multiplies the approximate heart rate by the obtained factor, and sets the resultant value as the setting time. For example, when an approximate heart rate of previous one pulse is 120 bpm and a factor corresponding to the approximate heart rate is 1.0, a setting time of 0.5 second is obtained. When the approximate heart rate of previous one pulse is 60 bpm, which is equal to the standard heart rate at rest, and a factor corresponding to the approximate heart rate is 1.5, a setting time of 1.5 seconds is obtained.

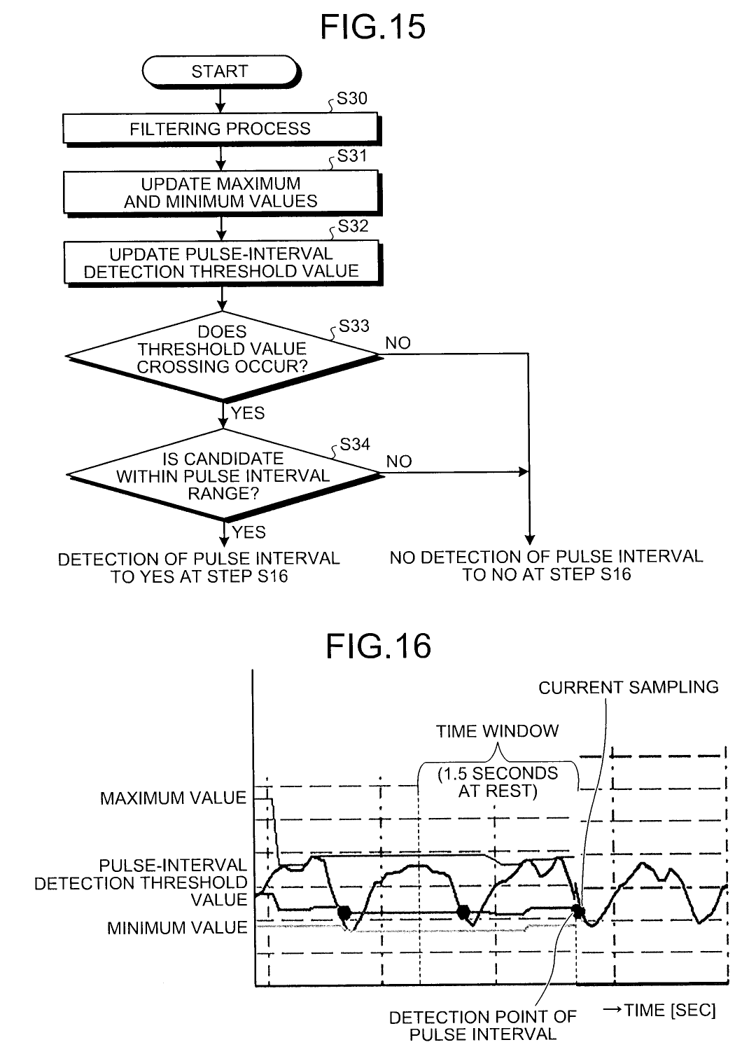

The biological information processing apparatus 100 then detects a pulse interval using the pulse signal outputted from the pulse-wave measuring unit 101, by means of the pulse-interval detecting unit 106 (step S15). FIG. 15 is a flowchart of a process procedure of detecting a pulse interval. The pulse-interval detecting unit 106 properly performs digital filtering with an FIR filter or the like according to filter characteristics depending on a hardware configuration of the pulse-wave measuring unit 101, and performs elimination of noise components other than a pulse wave (such as noises and fluctuations of a baseline) and steepening of the pulse waveform, using one of an LPF and a HPF or both thereof, as required (step S30). The pulse-interval detecting unit 106 then updates a maximum value and a minimum value of the pulse wave during a time window from a most recent sampling time up to a setting time (step S31). FIG. 16 is a drawing illustrating an example of a pulse wave during a time window from a most recent sampling time up to a setting time. As mentioned above, a setting time for a resting time is set at 1.5 seconds.

In the present embodiment, during rest immediately after an exercise, the pulse-interval detecting unit 106 updates the maximum and minimum values of the pulse wave using the setting time calculated at step S14, to change the setting time. The pulse-interval detecting unit 106 determines a pulse-interval detection threshold value (for example, a median of the maximum and minimum values) to be used for detection of crossing with the pulse wave (threshold value crossing) (step S32). Because characteristics of the wave (such as the form and the polarity) vary according to measuring systems, the pulse-interval detection threshold value is preferably set according to the measuring systems. This process allows easy dynamic follow-up to changes in the pulse wave amplitude.

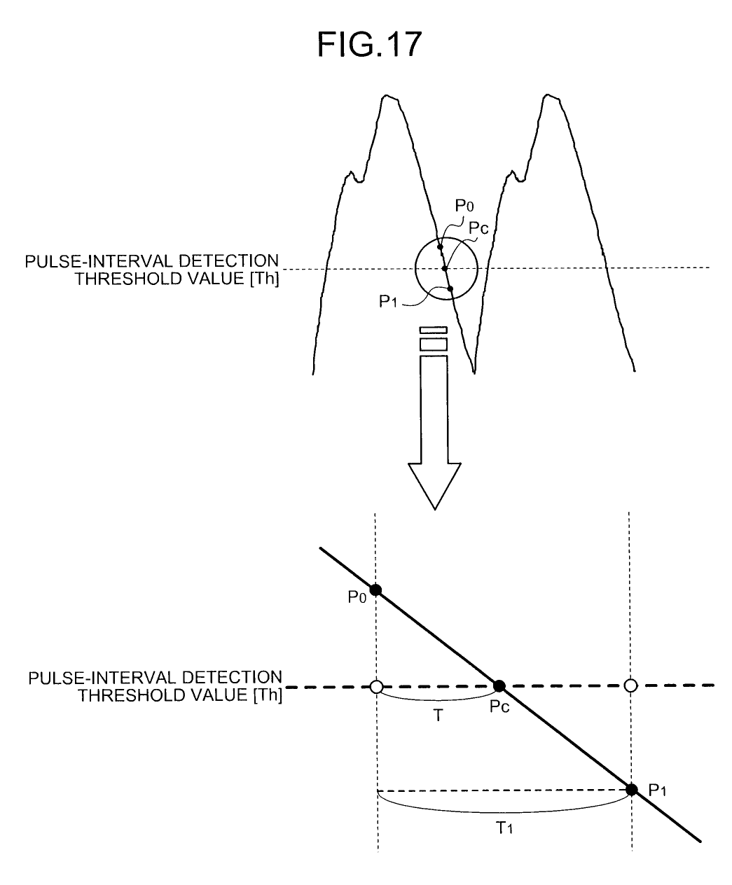

The pulse-interval detecting unit 106 then determines whether the pulse wave crosses the pulse-interval detection threshold value (in a direction previously determined), and determines a first sampling time when the pulse wave crosses the threshold value as a timing of detection of a pulse interval (step S33). Because the threshold value crossing occurs between samplings, there is a difference in the timing between sampling and actual threshold value crossing. Accordingly, the threshold value crossing can be subjected an approximating process to reduce influences of the difference. FIG. 17 is a drawing illustrating an example of the approximating process for the threshold value crossing. The approximating process as shown in FIG. 17 assumes that a pulse wave between samplings (between P0 and P1) is a straight line, and estimates threshold value crossing Pc using a ratio of amplitudes between before and after the pulse-interval detection threshold value (Th). In FIG. 17, T=T1×(P0−Th)/(P0−P1). The threshold value crossing Pc is calculated using T. A candidate for the pulse interval is thus detected; however, there are some cases in which noises are included or the pulse signal is not correctly measured. Accordingly, the pulse-interval detecting unit 106 determines whether the detected candidate for the pulse interval is within a pulse interval range previously set (for example, a range of pulse rates from 40 bpm to 120 bpm, that is, a range of pulse intervals from 0.5 second to 1.5 seconds) (step S34). When the detected candidate for the pulse interval is outside the pulse interval range (NO at step S34), the pulse-interval detecting unit 106 determines that no pulse interval is detected and that an error occurs. When the detected candidate for the pulse interval is within the pulse interval range (YES at step S34), the pulse-interval detecting unit 106 determines that a pulse interval is detected.

Return to the explanation of the pulse-interval detecting process with reference to FIG. 11. When the result of the determination at step S34 is YES and it is determined that a pulse interval is detected (YES at step S16), the biological information processing apparatus 100 proceeds to steps S17 to S19. When the result of the determination at step S34 is NO and it is determined that no pulse interval is detected and that an error occurs (NO at step S16), the biological information processing apparatus 100 returns to step S10.

The display unit 107 displays each pulse interval data indicating a result of the detection of the pulse interval at step S17, the communication unit 108 transmits each pulse interval data to an external information terminal at step S18, and the recording unit 109 temporarily stores the pulse interval data at step S19. The communication unit 108 can transfer the pulse interval data stored and accumulated by the recording unit 109 collectively to an external information terminal. When the measurement is completed (YES at step S20), the process terminates.

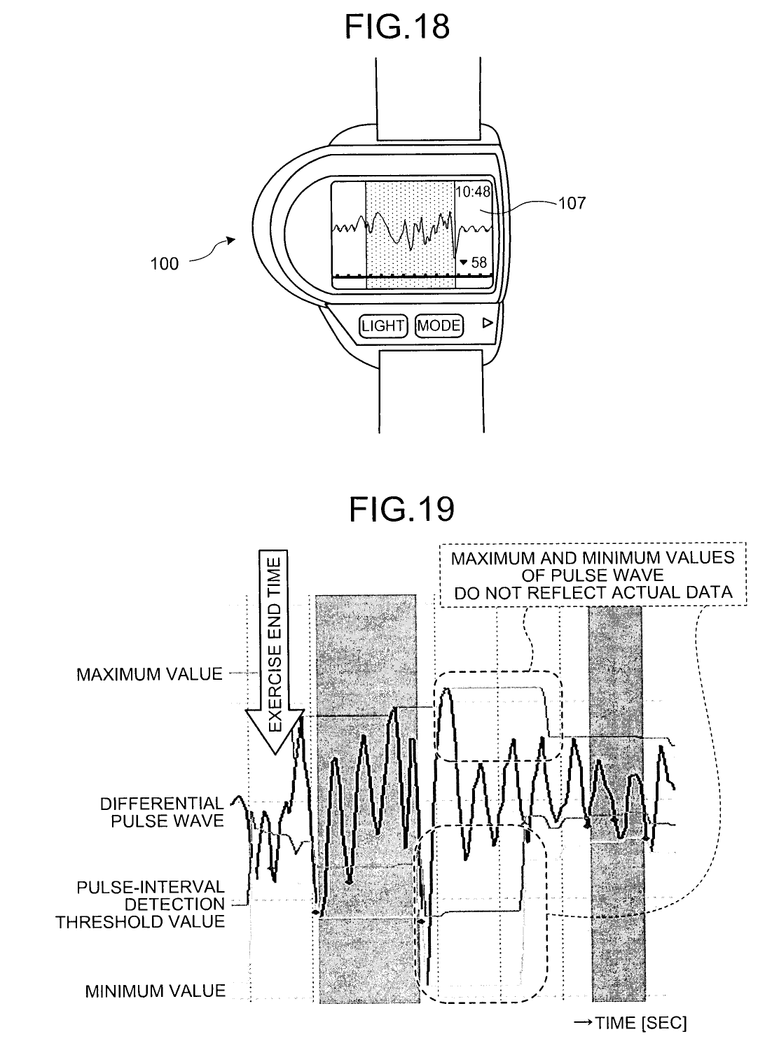

FIG. 18 is a drawing illustrating an example of display of the pulse interval data displayed on the display unit 107. A user can promptly see a result of the pulse interval detection on the biological information processing apparatus 100 that the user wears in the daytime, or can promptly see the pulse interval data transmitted by the communication unit 108 to a personal computer or a personal digital assistant. The user can obtain information such as a stress level and an exercise load at the measurement, as information that is secondarily obtained from the detection of the pulse interval.

With the configuration mentioned above, it is determined whether a user is during an exercising state or a resting state based on an average change rate of the body motion amount. An approximate heart rate is then calculated based on a result of the determination, a setting time is set using the approximate heart rate, and a pulse interval is detected. Accordingly, while the conventional pulse-wave detecting method that can highly accurately detect a pulse interval at rest is used as it is, a pulse interval at rest immediately after an exercise, which is conventionally difficult to detect, can be also detected with high accuracy.

The reason why the pulse interval during rest immediately after an exercise can be also detected with accuracy is as follows: During an exercising state, a pulse wave is made erratic due to body motion, so that a baseline or amplitude of the pulse wave frequently changes significantly. When for example 1.5 seconds is constantly used as the setting time for detection of a minimum value and a maximum value for calculating a pulse-interval detection threshold value to be used for detection of crossing with a pulse wave, a following problem can occur. FIG. 19 depicts a state of a pulse wave when a user shifts from an exercising state to a resting state. As shown in FIG. 19, during an exercising state, detection of the maximum and minimum values cannot follow abrupt changes in the amplitude or baseline of the pulse wave, so that a pulse-interval detecting threshold value that is not suitable for an actual waveform is calculated. Such erroneous detection can particularly occur for several seconds during rest immediately after an exercise. The setting time to be used for the detection of the minimum and maximum values from the pulse wave does not necessarily have be a fixed value of 1.5 seconds. The value of 1.5 seconds is based on a pulse rate of 60 bpm corresponding to one standard pulse at rest. This value is obtained by multiplying 60 bpm by 1.5 so that the obtained time surely includes one pulse. To detect a pulse interval in a case including an exercise time, it is appropriate that a setting time reflecting such physiological characteristics that the pulse quickens immediately after an exercise should be set. Thus, to reflect an exercise and the pulse physiological characteristics in detection of a pulse interval, an approximate heart rate is calculated based on information relating to an exercise including acceleration and a body motion amount at measurement, and a setting time is set using the calculated approximate heart rate, thereby detecting a pulse interval. Accordingly, erroneous detection of a pulse interval during rest immediately after an exercise can be particularly reduced.

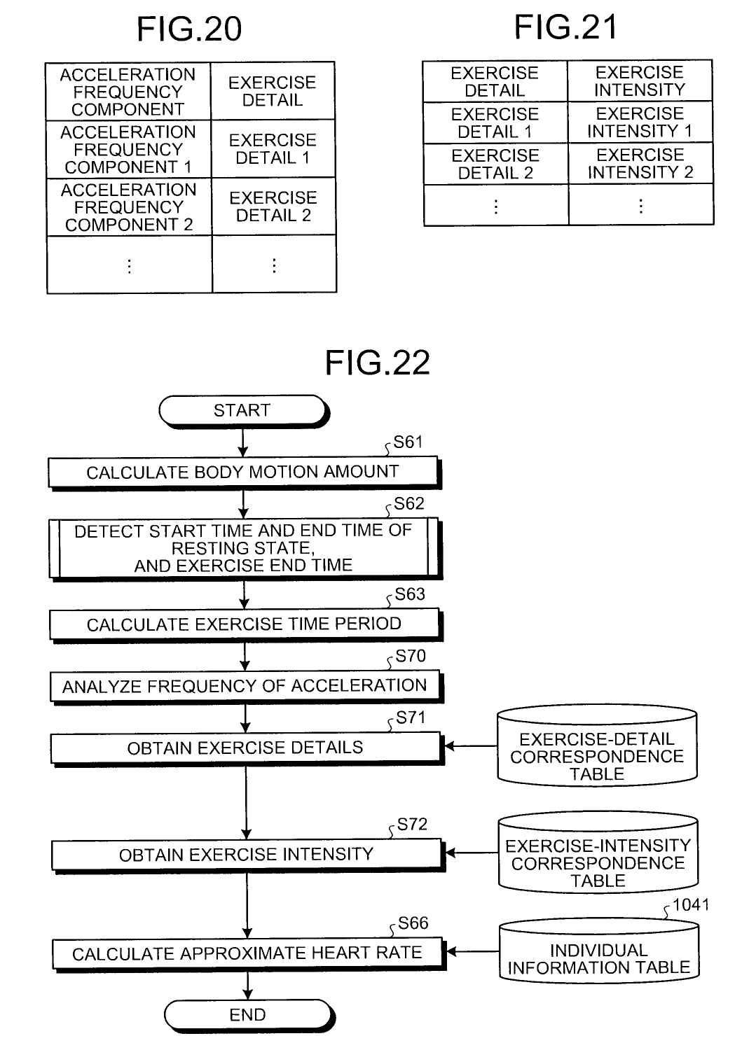

In the process at step S13 in the present embodiment, the approximate-heart-rate calculating unit 104 obtains exercise intensity corresponding to amplitude of an acceleration wave. Alternatively, the approximate-heart-rate calculating unit 104 can obtain exercise details and exercise intensity using frequency components of the acceleration. In this case, the biological information processing apparatus includes an exercise-detail correspondence table and a second exercise-intensity correspondence table (second correspondence information), instead of the exercise-intensity correspondence table 1040. FIG. 20 is a drawing illustrating an example of a data configuration of the exercise-detail correspondence table. The exercise-detail correspondence table provides a correspondence relation previously set between frequency components of acceleration and exercise details. Details of the correspondence relation are described for example in the reference literature 1. FIG. 21 is a drawing illustrating an example of a data configuration of the second exercise-intensity correspondence table. The second exercise-intensity correspondence table provides a correspondence relation between exercise details and exercise intensity. Details of the correspondence relation are described for example in the reference literature 2.

FIG. 22 is a flowchart of a process procedure of approximating a heart rate, for explaining details of the process at step S13 according to this modification (first modification). The processes from step S61 to step S63 are the same as those in the embodiment mentioned above. The approximate-heart-rate calculating unit 104 then analyzes a frequency of acceleration using the acceleration measured and outputted by the acceleration measuring unit 102 during the exercise time period calculated at step S62, to obtain frequency components of the acceleration (step S70). The approximate-heart-rate calculating unit 104 then obtains exercise details corresponding to the frequency components obtained at step S70, with reference to the exercise-detail correspondence table (step S71). The approximate-heart-rate calculating unit 104 further obtains exercise intensity corresponding to the exercise details obtained at step S71, with reference to the second exercise-intensity correspondence table (step S72). The approximate-heart-rate calculating unit 104 then calculates an approximate heart rate as approximation of the heart rate, using the obtained exercise intensity, the resting heart rate stored in the individual information table 1041, and the maximum heart rate calculated based on the individual information stored in the individual information table 1041, in the same manner as that in the embodiment described above (step S66).

It is known that the frequency components of the acceleration have peaks near 2 Hertz and 4 Hertz for example when a user is walking continuously for one minute at 3 km/h as the exercise details. Therefore, it is assumed that such a correspondence relation between the frequency components and the exercise details is stored in the exercise-detail correspondence table. It is also assumed that the exercise intensity corresponding to the exercise details, for example 30%, is stored in the second exercise-intensity correspondence table. When the user's pulse rate at rest is 60 bpm and the maximum heart rate is 190 bpm, an approximate heart rate of 69 bpm is calculated at step S66.

The approximate heart rate can be calculated also with the configuration mentioned above. By using the approximate heart rate, a pulse interval during rest immediately after an exercise can be also detected with high accuracy.

The information (second correspondence information) indicating the correspondence relation among the frequency components of the acceleration, the exercise details, and the exercise intensity is provided by two tables, that is, the exercise-detail correspondence table and the second exercise-intensity correspondence table. These two tables can be configured as one table.

In the process at step S13 in the embodiment mentioned above, the approximate-heart-rate calculating unit 104 can obtain a maximum volume of oxygen that can be taken into a body (VO2max) using the amplitude of the acceleration during an exercise. The approximate-heart-rate calculating unit 104 can obtain an approximate heart rate based on a HR-VO2max relation (see the reference literature 3). In this case, the biological information processing apparatus includes an energy-expenditure correspondence table and a VO2max correspondence table (third correspondence information), instead of the exercise-intensity correspondence table 1040. The energy-expenditure correspondence table provides a correspondence relation previously set between the amplitude of the acceleration wave and the energy expenditure. Details of the correspondence relation are described for example in the reference literature 3. The VO2max correspondence table provides a correspondence relation between the energy expenditure and VO2max. Details of the correspondence relation are described for example in the reference literature 2. Other than the reference literatures 2 and 3, the following reference literature 4 can be also referred. (Reference Literature 4) Estimation of energy expenditure by a portable accelerometer. Medicine and Science in sports and exercise 15(5) 403-407.

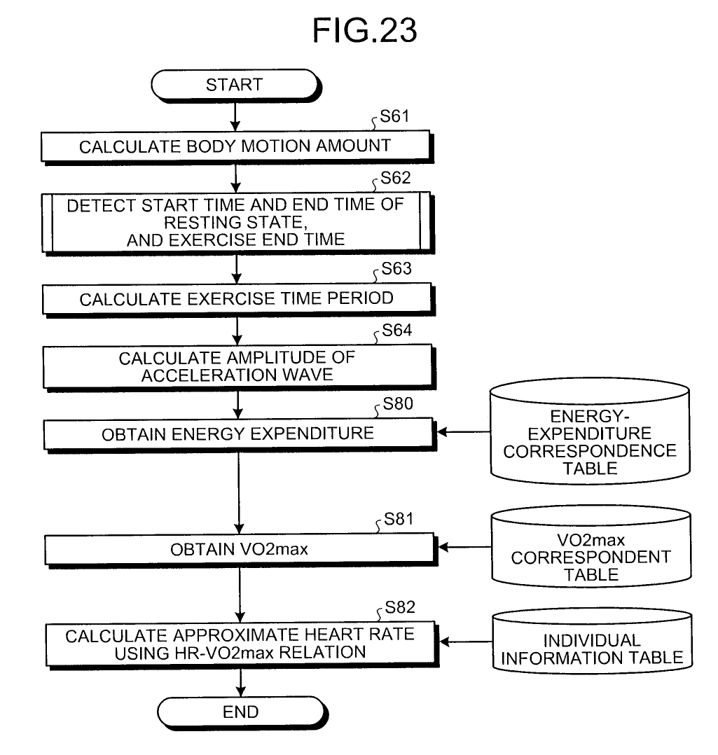

FIG. 23 is a flowchart of a process procedure of approximating a heart rate, for explaining details of the process at step S13 according to this modification (second modification). The processes from step S61 to step S64 are the same as those in the embodiment described above. The approximate-heart-rate calculating unit 104 then obtains energy expenditure corresponding to the amplitude obtained at step S64, with reference to the energy-expenditure correspondence table (step S80). The approximate-heart-rate calculating unit 104 further obtains VO2max corresponding to the energy expenditure obtained at step S80, with reference to the VO2max correspondence table (step S81). The approximate-heart-rate calculating unit 104 then calculates an approximate heart rate according to the HR-VO2max relation using VO2max obtained at step S81, the resting heart rate stored in the individual information table 1041, and the maximum heart rate calculated based on the individual information stored in the individual information table 1041 (step S82).

Also with this configuration, an approximate heart rate can be calculated, and a pulse interval at rest immediately after an exercise can be detected with high accuracy using the calculated approximate heart rate.

The information (third correspondence information) indicating a correspondence relation among the amplitude of the acceleration, the energy expenditure, and the maximum oxygen intake is provided by two tables of the energy-expenditure correspondence table and the VO2max correspondence table. However, these two tables can be configured as one table.

It is also possible to approximate a heart rate by another method using at least one of the acceleration and the body motion amount.

In the embodiment mentioned above, the biological information processing apparatus 100 includes the exercise-intensity correspondence table 1040 and the individual information table 1041. However, the biological information processing apparatus 100 can include neither the exercise-intensity correspondence table 1040 nor the individual information table 1041, and properly obtain information stored in the exercise-intensity correspondence table 1040 and the individual information table 1041 that are included in an external device.

Also in the first modification, the biological information processing apparatus can include none of the individual information table 1041, the exercise-detail correspondence table, and the second exercise-intensity correspondence table, and properly obtain information stored in these tables that are included in an external device.

Also in the second modification, the biological information processing apparatus can include none of the individual information table 1041, the energy-expenditure correspondence table, and the VO2max correspondence table, and properly obtain information stored in these tables that are included in an external device.

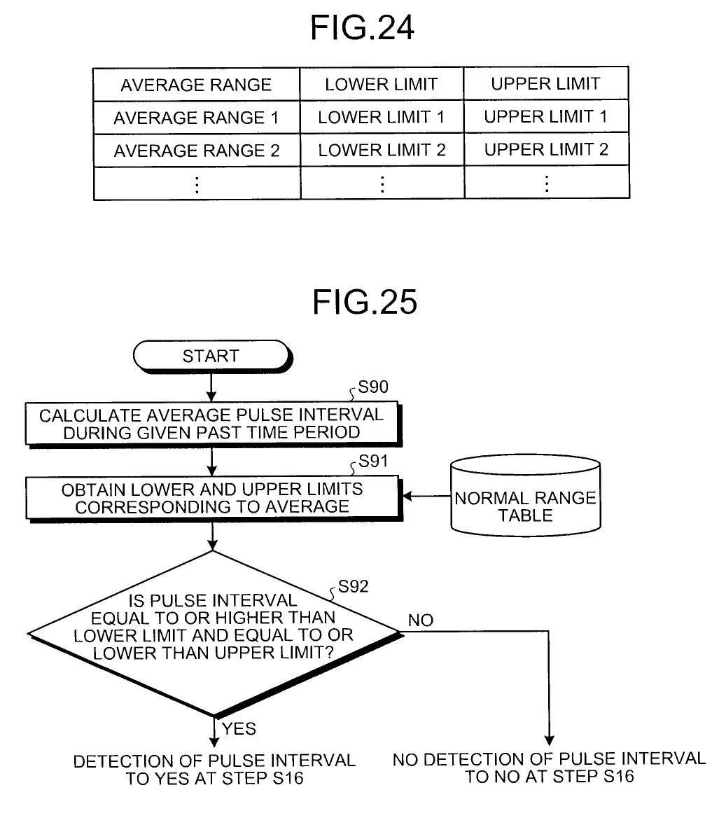

At step S34 in the embodiment mentioned above, the pulse-interval detecting unit 106 determines whether the candidate for the pulse interval detected at step S33 is within the pulse interval range previously set. The pulse-interval detecting unit 106 can determine whether the candidate for the pulse interval is within a normal range, using an average of the pulse intervals. In this modification (third modification), the biological information processing apparatus further includes a normal range table. FIG. 24 is a drawing illustrating an example of a data configuration of the normal range table. The normal range table provides a correspondence relation previously set between a range of average pulse intervals and upper and lower limits of the pulse interval as normal ranges. FIG. 25 is a flowchart of a process procedure of determining whether a pulse interval for which a result of determination at step S34 is YES is erroneous. For the pulse interval for which the result of the determination at step S34 is YES, the pulse-interval detecting unit 106 calculates an average of the pulse intervals during a given past period of time (step S90). The pulse-interval detecting unit 106 then obtains lower and upper limits corresponding to the average calculated at step S90, with reference to the normal range table (step S91). The pulse-interval detecting unit 106 determines whether the pulse interval for which the result of the determination at step S34 is YES is equal to or higher than the lower limit, and equal to or lower than the upper limit, the lower and upper limits being obtained at step S91 (step S92). When a result of the determination at step S92 is YES, the pulse-interval detecting unit 106 determines that a pulse interval is detected. When a result of the determination at step S92 is NO, the pulse-interval detecting unit 106 determines that no pulse interval is detected and that an error occurs.

With this configuration, a pulse interval during an exercising state in which the body motion amount calculated by the body-motion calculating unit 103 is particularly large comes to be determined erroneous even when the detection is performed.

Both of the upper and lower limits of the pulse interval are used as the normal range; however, at least one of the upper and lower limits can be used. In this case, a correspondence relation between the range of the average pulse intervals and at least one of the upper and lower limits of the pulse interval is previously set in the normal range table.

At step S34, the pulse-interval detecting unit 106 can determine whether the candidate for the pulse interval detected at step S33 is erroneous, based on the body motion amount calculated at step S61. In this modification (fourth modification), the normal range table previously stores therein, for example, at least one of upper and lower limits of the body motion amount. When the body motion amount calculated at step S61 is at least either lower than the lower limit or higher than the upper limit stored in the normal range table, the pulse-interval detecting unit 106 determines that the candidate for the pulse interval for which the result of the determination at step S34 is YES is erroneous, and determines that no pulse interval is detected.

The lower and upper limits can be changed using the approximate heart rate. For example when an upper limit of 150 bpm is initially set, and then when an average pulse interval for a given period of time, which is obtained by using data of pulse intervals previously detected, exceeds the upper limit of 150 bpm, the setting of the upper limit can be changed to the user's maximum heart rate. It is also possible to update the lower and upper limits in combination with the exercise details obtained in the process of calculating the approximate heart rate. The settings of details of an exercise and the upper and lower limits of the heart rate in a state where a user is doing the exercise can be updated for each user.

In the embodiment as mentioned above, the biological information processing apparatus 100 includes the display unit 107, the communication unit 108, and the recording unit 109, as outputting means. However, according to another modification (fifth modification), the biological information processing apparatus 100 does not have to include these units, or can include at least one of these units. When the biological information processing apparatus 100 includes the display unit 107 and the communication unit 108, the communication unit 108 does not have to immediately transfer the pulse interval data to an external information terminal.

According to still another modification (sixth modification), the biological information processing apparatus can further include a converting unit that converts the pulse interval detected by the pulse-interval detecting unit 106 into a pulse rate. The biological information processing apparatus according to the sixth modification can be adapted to output the pulse rate obtained by the converting unit to at least one of the display unit 107, the communication unit 108, and the recording unit 109.

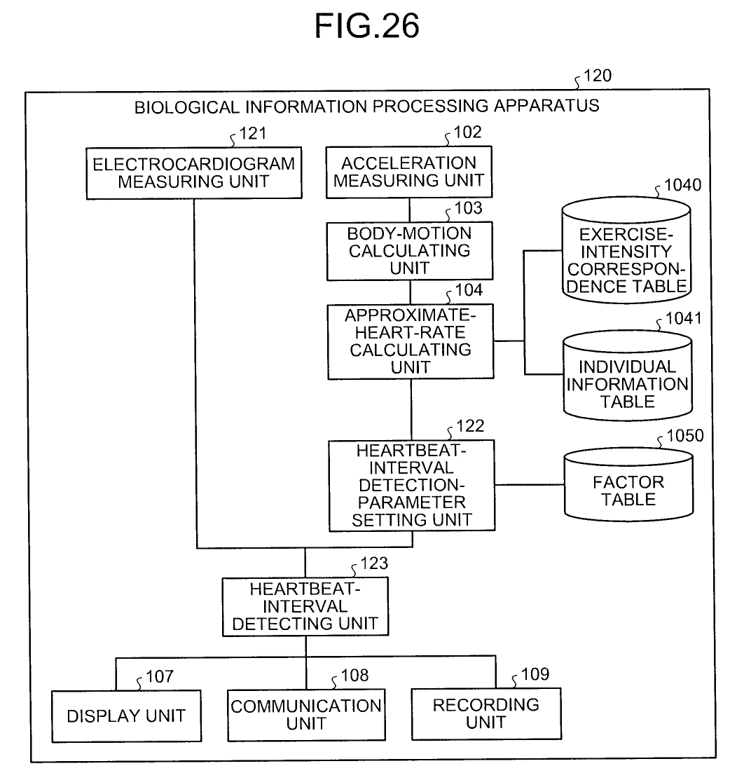

In the embodiment as mentioned above, the biological information processing apparatus 100 includes the pulse-wave measuring unit 101 that measures a pulse wave, as a unit for measuring heartbeats. However, the biological information processing apparatus can be adapted to include an electrocardiogram measuring unit that measures an electrocardiogram, instead of the pulse-wave measuring unit 101. FIG. 26 is a drawing illustrating an example of a configuration of a biological information processing apparatus 120 according to this modification (seventh modification). The biological information processing apparatus 120 is different from the biological information processing apparatus 100 according to the embodiment as mentioned above in a following respect. The biological information processing apparatus 120 includes an electrocardiogram measuring unit 121, a heartbeat-interval detection-parameter setting unit 122, and a heartbeat-interval detecting unit 123, instead of the pulse-wave measuring unit 101, the pulse-interval detection-parameter setting unit 105, and the pulse-interval detecting unit 106. The factor table 1050 stores therein a correspondence relation between factors to be used for calculation of the setting time that is used for detection of a heartbeat interval rather than the pulse-interval, and ranges of heart rates.

The heartbeat-interval detecting unit 123 obtains a heartbeat-interval detection threshold value using a maximum value and a minimum value of a waveform of an electrocardiogram during a time window from a most recent sampling time up to the setting time. The heartbeat-interval detecting unit 123 then detects a detection point of a heartbeat interval corresponding to each heartbeat using the obtained heartbeat-interval detection threshold value, thereby detecting a heartbeat interval. In this seventh modification, the heartbeat-interval detecting unit 123 uses a setting time calculated by the heartbeat-interval detection-parameter setting unit 122. Similarly the pulse-interval detection-parameter setting unit 105 as mentioned above, the heartbeat-interval detection-parameter setting unit 122 obtains a factor corresponding to an approximate heart rate calculated by the approximate-heart-rate calculating unit 104, with reference to the factor table 1050, and calculates a setting time using the obtained factor. The configuration of the biological information processing apparatus 120 other than these units is approximately the same as that of the embodiment as mentioned above, and thus the explanation thereof is omitted.

With the configuration mentioned above, the heartbeat interval can be detected with high accuracy also during rest immediately after an exercise.

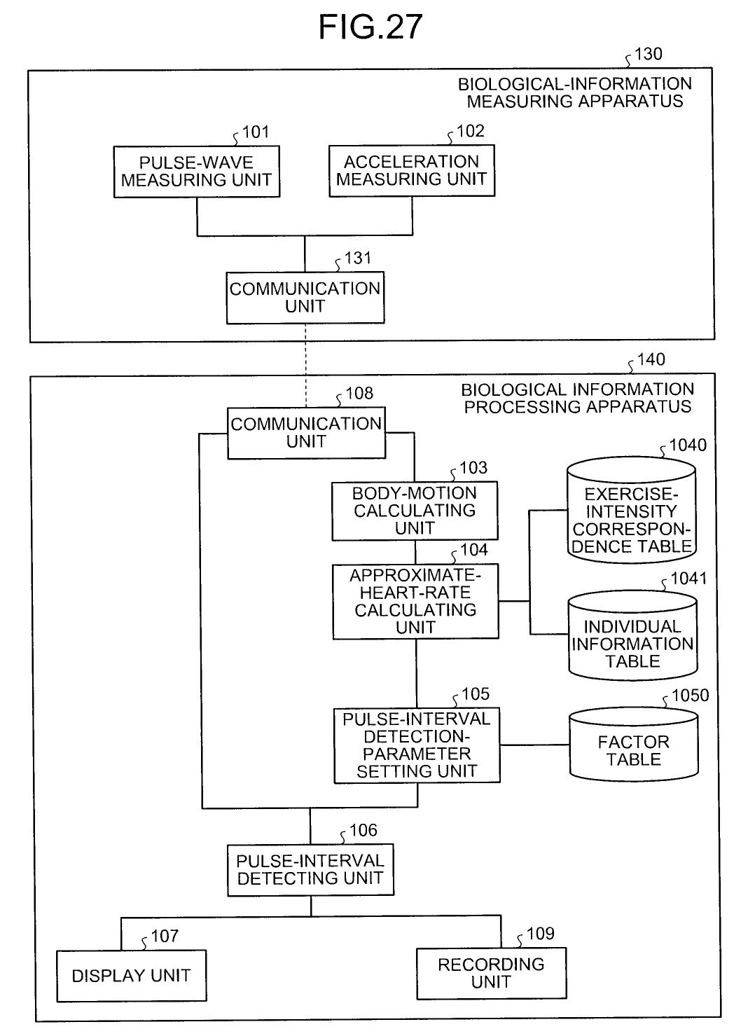

In the embodiment as mentioned above, the biological information processing apparatus 100 includes the pulse-wave measuring unit 101 and the acceleration measuring unit 102 to provide a function of an apparatus that measures biological information. However, the biological information processing apparatus 100 can eliminate the pulse-wave measuring unit 101 and the acceleration measuring unit 102, and can be adapted to obtain a pulse wave signal and acceleration from an external device. FIG. 27 is a drawing illustrating an example of a configuration of a biological information processing apparatus 140 according to this modification (eighth modification), and a configuration of a biological-information measuring apparatus 130 as an external device. The biological-information measuring apparatus 130 includes the pulse-wave measuring unit 101, the acceleration measuring unit 102, and a communication unit 131 that is configured by a network interface or the like. The biological information processing apparatus 140 receives a pulse wave signal and acceleration from the biological-information measuring apparatus 130 via the communication unit 108. The biological information processing apparatus 140 detects a pulse interval using the received pulse wave signal in the same manner as that in the embodiment as described above.

This configuration enables a computer having a typical hardware configuration, for example, to be used as the biological information processing apparatus 140, so that biological information measured by the biological-information measuring apparatus 130 can be analyzed efficiently.

In the eighth modification, the pulse-wave measuring unit 101 and the acceleration measuring unit 102 are installed in one biological-information measuring apparatus 130; however, the pulse-wave measuring unit 101 and the acceleration measuring unit 102 can be separate measuring apparatuses. In such a case, the biological information processing apparatus 140 can obtain a pulse wave signal and acceleration from the separate measuring apparatuses, respectively.

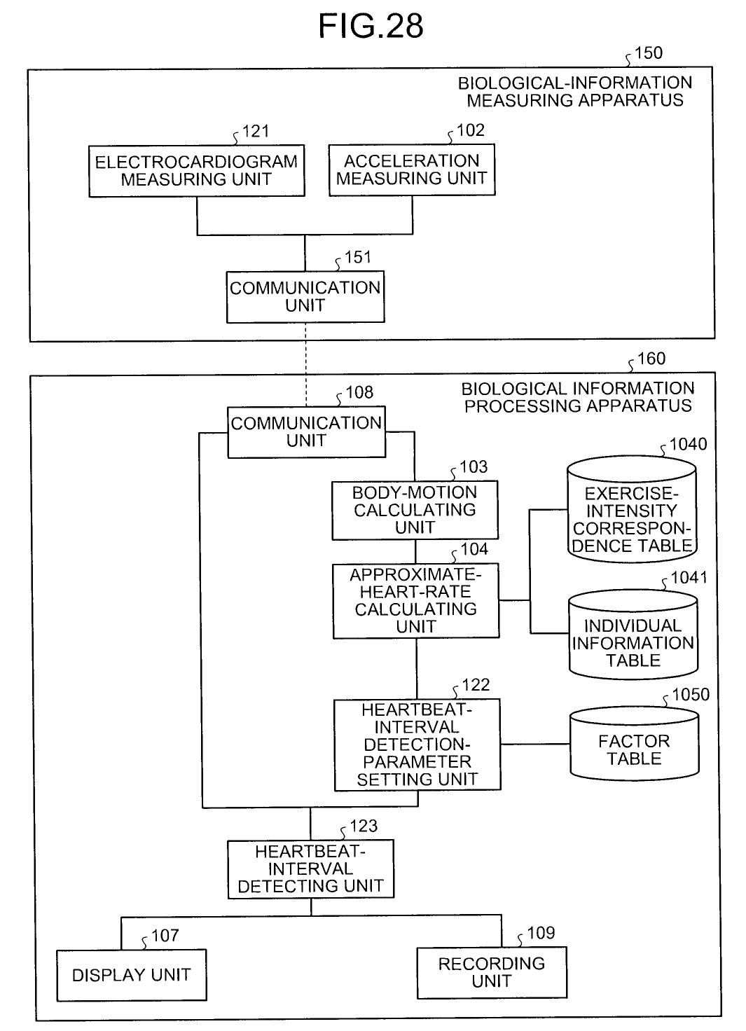

The biological information processing apparatus 120 according to the seventh modification includes the electrocardiogram measuring unit 121 and the acceleration measuring unit 102 to provide a function of an apparatus that measures biological information. However, the biological information processing apparatus 120 can similarly eliminate these units, and can obtain an electrocardiographic signal and acceleration from an external device. FIG. 28 is a drawing illustrating an example of a biological information processing apparatus 160 according to this modification (ninth modification), and a configuration of a biological-information measuring apparatus 150 as an external device. The biological-information measuring apparatus 150 includes the electrocardiogram measuring unit 121, the acceleration measuring unit 102, and a communication unit 151 that is configured by a network interface or the like. The biological-information measuring apparatus 150 transmits an electrocardiographic signal measured by the electrocardiogram measuring unit 121 and acceleration measured by the acceleration measuring unit 102, to the biological information processing apparatus 160 via the communication unit 151. The biological information processing apparatus 160 receives the electrocardiographic signal and the acceleration from the biological-information measuring apparatus 150 via the communication unit 108. The biological information processing apparatus 160 detects a heartbeat interval using the received electrocardiographic signal in the same manner as that in the seventh modification.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A biological information processing apparatus comprising:
an obtaining unit that obtains a pulse wave signal indicating a pulse wave of a subject and an acceleration measured according to body motion of the subject;
a body-motion calculating unit that calculates an amount of body motion of the subject using the acceleration;
an approximating unit that approximates a heart rate of the subject using at least one of the body motion amount and the acceleration;
a setting unit that sets a parameter to be used for detection of a pulse interval, using the heart rate; and
a detecting unit that detects each pulse interval using a pulse waveform indicated by the pulse wave signal and the parameter.

2. The apparatus according to claim 1, wherein the approximating unit includes
a time calculating unit that detects an end time of an exercising state and a start time of the exercising state of the subject using the acceleration and the body motion amount, and calculates an exercise time period from the start time of the exercising state to the end time of the exercising state, and
an exercise approximating unit that approximates the heart rate using at least one of the acceleration measured during the exercise time period and the body motion amount calculated using the acceleration.

3. The apparatus according to claim 2, wherein the exercise approximating unit includes
an exercise analyzing unit that obtains exercise intensity corresponding to the obtained acceleration as exercise information, using first correspondence information indicating a correspondence relation between amplitude of acceleration and exercise intensity, and
a first approximating unit that approximates the heart rate using the exercise information.

4. The apparatus according to claim 2, wherein
the exercise approximating unit includes
an exercise analyzing unit that obtains exercise intensity corresponding to the obtained acceleration as exercise information, using second correspondence information indicating a correspondence relation among frequency components of acceleration, details of exercises, and exercise intensity, and
a first approximating unit that approximates the heart rate using the exercise information.

5. The apparatus according to claim 2, wherein
the exercise approximating unit includes
an exercise analyzing unit that obtains a maximum oxygen intake corresponding to the obtained acceleration as exercise information, using third correspondence information indicating a correspondence relation among amplitude of acceleration, energy expenditure, and maximum oxygen intake, and
a first approximating unit that approximates the heart rate using the exercise information.

6. The apparatus according to claim 3, wherein the first approximating unit obtains a maximum heart rate of the subject using individual information including at least one of age, sex, weight, and a heart rate at rest of the subject, and approximates the heart rate using the maximum heart rate and the exercise information.

7. The apparatus according to claim 1, wherein
the setting unit obtains a factor for changing a setting time according to the heart rate approximated by the approximating unit, calculates the setting time using the obtained factor, and sets the setting time as the parameter,
the factor decreases the setting time for a range of higher heart rates, and increases the setting time for a range of lower heart rates, and
the detecting unit calculates a pulse-interval detection threshold value using a maximum value and a minimum value of the pulse wave indicated by the pulse wave signal obtained during a time period from a most recent point of time when the pulse wave signal is obtained up to the setting time set as the parameter, and detects a detection point of the pulse interval corresponding to each pulse using the pulse-interval detection threshold value.

8. The apparatus according to claim 7, wherein the setting unit obtains the factor corresponding to the heart rate approximated by the approximating unit, using fourth correspondence information indicating a correspondence relation between ranges of heart rates and the factors, calculates the setting time using the factor, and sets the calculated setting time as the parameter.

9. A biological information processing apparatus comprising:
an obtaining unit that obtains an electrocardiograph signal indicating an electrocardiogram of a subject and an acceleration measured according to body motion of the subject;
a body-motion calculating unit that calculates an amount of body motion of the subject using the acceleration;
an approximating unit that approximates a heart rate of the subject using at least one of the body motion amount and the acceleration;
a setting unit that sets a parameter to be used for detection of a heart rate interval, using the heart rate; and
a detecting unit that detects each heart rate interval using an electrocardiogram waveform indicated by the electrocardiograph signal and the parameter.

10. A biological-information processing method performed by a biological information processing apparatus including an obtaining unit, a body-motion calculating unit, an approximating unit, a setting unit, and a detecting unit, the method comprising:
obtaining a pulse wave signal indicating a pulse wave of a subject, and an acceleration measured according to body motion of the subject, by the obtaining unit;
calculating an amount of body motion of the subject using the acceleration, by the body-motion calculating unit;
approximating a heart rate of the subject using at least one of the body motion amount and the acceleration, by the approximating unit;
setting a parameter to be used for detection of a pulse interval using the heart rate, by the setting unit; and
detecting each pulse interval using a pulse waveform indicated by the pulse wave signal and the parameter, by the detecting unit.

11. A biological-information processing method performed by a biological information processing apparatus including an obtaining unit, a body-motion calculating unit, an approximating unit, a setting unit, and a detecting unit, the method comprising:
obtaining an electrocardiograph signal indicating an electrocardiogram of a subject, and an acceleration measured according to body motion of the subject, by the obtaining unit;
calculating an amount of body motion of the subject using the acceleration, by the body-motion calculating unit;
approximating a heart rate of the subject using at least one of the body motion amount and the acceleration, by the approximating unit;
setting a parameter to be used for detection of a heart rate interval using the heart rate, by the setting unit; and
detecting each heart rate interval using an electrocardiogram waveform indicated by the electrocardiograph signal and the parameter, by the detecting unit.
